(19) United States
(12) Patent Application Publication
(10) Pub. No.: US 2016/0324573 A1
(43) Pub. Date: Nov. 10, 2016

(54) DEVICES AND METHODS FOR DELIVERING THERAPEUTIC ELECTRICAL IMPULSES

(71) Applicant: IOWA APPROACH INC., Menlo Park, CA (US)

(72) Inventors: Steven R. MICKELSON, Iowa City, IA (US); Raju VISWANATHAN, Mountain View, CA (US); Gary LONG, Cincinnati, OH (US)

(73) Assignee: IOWA APPROACH INC., MENLO PARK, CA (US)

(21) Appl. No.: 15/201,983

(22) Filed: Jul. 5, 2016

Related U.S. Application Data
(63) Continuation of application No. PCT/US2015/010138, filed on Jan. 5, 2015.
(60) Provisional application No. 61/923,971, filed on Jan. 6, 2014.

(51) Int. Cl. A61B 18/14

(52) U.S. Cl. CPC A61B 18/1492; A61B 2018/1435; A61B 2018/00577

(57) ABSTRACT

An apparatus includes an electrode including a first electrode portion and a second electrode portion. The first electrode portion and the second electrode portion collectively form an outer surface from which an electric field is produced when a voltage is applied to the electrode. The first electrode portion is constructed from a first material having a first electrical conductivity. The second electrode portion is distinct from the first electrode portion, and is constructed from a second material. The second material has a second electrical conductivity that is different than the first electrical conductivity.

### CROSS-REFERENCE TO RELATED APPLICATIONS

This application claims benefit of priority to U.S. Provisional Application Ser. No. 61/923,971, entitled “Composite Electrode Design to Reduce Probability of Flash Arcing in High Voltage Electrical Impulse Delivery,” filed Jan. 6, 2014, which is incorporated herein by reference in its entirety.

### BACKGROUND

The embodiments described herein relate generally to medical devices for therapeutic electrical energy delivery, and more particularly to electrodes for delivering electrical impulses for selective irreversible electroporation.

The past two decades have seen advances in the technique of electroporation as it has progressed from the laboratory to clinical applications. Known methods include applying brief, high voltage DC pulses to tissue, thereby generating locally high electric fields, typically in the range of hundreds of Volts/centimeter. The electric fields disrupt cell membranes by generating pores in the cell membrane, which subsequently destroys the cell membrane and the cell. While the precise mechanism of this electrically-driven pore generation (or electroporation) awaits a detailed understanding, it is thought that the application of relatively large electric fields generates instabilities in the phospholipid bilayers in cell membranes, as well as mitochondria, causing the occurrence of a distribution of local gaps or pores in the membrane. If the applied electric field at the membrane exceeds a threshold value, typically dependent on cell size, the electroporation is irreversible and the pores remain open, permitting exchange of material across the membrane and leading to apoptosis or cell death. Subsequently, the surrounding tissue heals in a natural process.

Some known tissue ablation methods employ irreversible electroporation for the purpose of treating tumors by exposing them to high levels of DC voltage. Such known methods of treating tumors typically involve destroying a significant mass of tissue. Such known methods can also produce high temperatures (i.e., that exceed desired limits) within the target and/or surrounding tissue.

Known catheters with multiple electrodes have been used to produce irreversible electroporation to ablate cardiac tissue for the treatment of cardiac arrhythmias, such as atrial fibrillation. While pulsed DC voltages are known to drive electroporation under certain circumstances, known delivery methods and systems do not provide specific means of limiting possible damage to nearby tissue when the target tissue to be ablated is relatively further away. For example, in some situations, high voltages at the electrodes can result in flash arcing or electrical discharges around portions of an electrode. In such situations, localized electric field intensities can be large enough to produce undesirable dielectric breakdown and/or to generate electrical discharges or sparking, causing local thermal damage and possible charring debris.

Moreover, regions of high curvature in the geometry of known electrodes (e.g., the curvature towards the ends of a ring electrode) are prone to arcing. Specifically, the geometry of the electrode can influence the spatial distribution of local electric field intensity near the electrode. Thus, some known electrodes are designed to minimize electrode surface curvature by rounding edges. However, there are practical limits to such approaches of adjusting the electrode geometry, especially when high voltages are desired.

Thus, a need exists for improved methods and devices for safer and more selective energy delivery methods to produce tissue ablation at a target tissue location, while leaving surrounding tissue elsewhere relatively intact and unchanged. Similarly stated, a need exists for improved methods and devices for generating a local electric field in a tissue region that is large enough to drive irreversible electroporation in that region, while maintaining electric field values below a safe level in that tissue region and surrounding tissue regions. A need exists for systems and methods that avoid the generation of dielectric breakdown during delivery of therapeutic electrical impulses.

### SUMMARY

The embodiments of the present disclosure include devices and methods for selective application of electroporation therapy in a minimally invasive context while suppressing the generation of undesirable electrical discharge or breakdown. The embodiments described herein can result in well-controlled and specific delivery of electroporation in a safe and efficacious manner while preserving overall tissue integrity. In some embodiments, an apparatus includes an electrode including a first electrode portion and a second electrode portion. The first electrode portion and the second electrode portion collectively form an outer surface from which an electric field is produced when a voltage is applied to the electrode. The first electrode portion is constructed from a first material having a first electrical conductivity. The second electrode portion is distinct from the first electrode portion, and is constructed from a second material. The second material has a second electrical conductivity that is different than the first electrical conductivity.

### BRIEF DESCRIPTION OF THE DRAWINGS

FIG. 1 is a schematic illustration showing a first electrode in the form of an annular ring disposed with a surrounding tissue environment and a surface representing a second electrode, where a voltage applied between the electrodes results in current flow to the second electrode from and through the first electrode and the tissue environment.

FIG. 2 is a schematic illustration of an electrode according to an embodiment with an annular cross section including two distinct materials with different electrical conductivities.

FIG. 3A is a schematic illustration of a catheter electrode according to an embodiment abutting a catheter shaft and showing a local geometry for local boundary field analysis.

FIG. 3B is a cross-sectional view of a portion of an electrode according to an embodiment coupled to a catheter shaft.

FIG. 4 is a perspective view of a ring-shaped (with annular cross-section) composite electrode according to an embodiment, including a higher electrical conductivity region disposed between two regions having a relatively lower electrical conductivity.

FIG. 5 shows a perspective view of a composite electrode according to an embodiment, where the electrode has a uniform outer surface.

FIG. 6 illustrates a composite electrode according to an embodiment, including a section constructed from a first material with a first electrical conductivity that is plated or deposited over a second material with a second electrical conductivity.

FIG. 7 is a perspective view of a composite electrode according to an embodiment, having a midsection comprising a rigid electrode in the form of a cylindrical annular electrode disposed between two flexible coil end sections.

FIG. 8 is a perspective view of a composite electrode according to an embodiment, having a midsection constructed from a coil disposed between two flexible coil end sections constructed from a different material.

FIG. 9 is an illustration of an annular electrode according to an embodiment including a first annular electrical conductor abutting a second annular electrical conductor.

FIGS. 10A-C show a top view, front side view and right side view, respectively, of a composite electrode according to an embodiment.

FIG. 11 is a perspective view of a composite electrode according to an embodiment with segments of different materials.

FIG. 12 shows a comparison chart of peak electric field values at the edges and at the lateral surface of an electrode according to an embodiment and a single-material electrode.

FIG. 13 is perspective view of a distal portion of a flexible medical device including a series of electrodes according to an embodiment.

FIGS. 14A-C show a top view, front side view and right side view, respectively, of a composite electrode according to an embodiment.

FIGS. 15A and 15B are a front view and a side view, respectively, of an electrode according to an embodiment.

FIG. 16 is a side view of a portion of a medical device including an electrode according to an embodiment.

FIG. 17 is a side view of a portion of a medical device including an electrode according to an embodiment.

FIG. 18 is a side view of a portion of a medical device including an electrode according to an embodiment.

FIG. 19 is a flowchart illustrating a method of delivering electric impulse therapy according to an embodiment.

### DETAILED DESCRIPTION

Devices for delivering electrical impulses are described herein. In some embodiments, an electrode is configured to produce an electric field having improved spatial uniformity (i.e., the difference between the average and the peak electric field values is reduced when compared to that from known systems or methods) by using geometric considerations together with composite and/or multiple different materials. In some embodiments, the electrode surfaces include at least two different materials with differing values of electrical conductivity. The portion of the electrode material surface with a relatively smaller electrical conductivity also includes regions of relatively larger curvature (such as edges), while the portion of the electrode surface with a relatively larger electrical conductivity includes regions of relatively smaller (or less) curvature. By combining the effects of geometric curvature and electrical conductivity in this manner, zones with large and/or discontinuous changes in electrical conductivity (e.g., between the tissue and the electrode), particularly in regions with relatively larger curvature, are minimized. Accordingly, the embodiments described herein can minimize the peak electric field intensity, which can often be higher in regions where the electrical conductivity sees large transitions and/or regions of where the electrode surface is discontinuous and/or has a high rate of curvature.

In some embodiments, an apparatus includes catheter devices for the selective and rapid application of DC voltage to produce electroporation. The catheter device has a set of composite (or “multi-material”) electrodes for ablation or delivery of voltage pulses. The voltage pulses can, for example, have pulse widths in the range of tens to hundreds of microseconds. In some embodiments, there could be a multiplicity of such voltage pulses applied through the electrodes, with an interval between pulses that can, for illustrative purposes, be in the range of tens to hundreds of microseconds. The composite and/or multi-material electrodes can be constructed from a range of materials, and have any suitable geometries and constructions disclosed herein that result in reduction of peak electric field intensities and minimized likelihood of flash arcing

In some embodiments, an apparatus includes an electrode including a first electrode portion and a second electrode portion. The first electrode portion and the second electrode portion collectively form an outer surface from which an electric field is produced when a voltage is applied to the electrode. The first electrode portion is constructed from a first material having a first electrical conductivity. The second electrode portion is distinct from the first electrode portion, and is constructed from a second material. The second material has a second electrical conductivity that is different than the first electrical conductivity.

In some embodiments, an apparatus includes a ring electrode configured to be coupled to a catheter shaft. The ring electrode includes a first electrode portion and a second electrode portion that collectively form a cylindrical outer surface from which an electric field is produced when a voltage is applied to the electrode. The second electrode portion forms at least a portion of an end surface configured to be coupled to the catheter shaft. The first electrode portion is constructed from a first material having a first electrical conductivity, and the second electrode portion is constructed from a second material. The second material has a second electrical conductivity different than the first electrical conductivity.

In some embodiments, an apparatus includes an electrode configured to be coupled to a catheter shaft. The electrode includes a first electrode portion and a second electrode portion, from which an electric field is produced when a voltage is applied to the electrode. At least the first electrode portion and the second electrode portion collectively form an outer surface. At least one of the first electrode portion or the second electrode portion include a flexible coil. The first electrode portion is constructed from a first material having a first electrical conductivity. The second electrode portion is constructed from a second material having a second electrical conductivity different than the first electrical conductivity.

In some embodiments, an apparatus includes an electrode including a first electrode portion and a second electrode portion. The first electrode portion has a first surface, and the second electrode portion has a second surface. The first surface is recessed from the second surface. The first surface and the second surface collectively form an outer surface from which an electric field is produced when a voltage is applied to the electrode. The first electrode portion is constructed from a first material having a first electrical conductivity. The second electrode portion is constructed from a second material having a second electrical conductivity different than the first electrical conductivity.

In some embodiments, an apparatus includes an electrode configured to be coupled to a medical device. The electrode includes a first electrode portion and a second electrode portion. The first electrode portion and the second electrode portion collectively form an outer surface from which an electric field is produced when a voltage is applied to the electrode. The first electrode portion has an outer diameter that varies along a longitudinal axis of the medical device. The first electrode portion is constructed from a first material having a first electrical conductivity. The second electrode portion is coupled to the first electrode portion along a surface defining the outer diameter. The second electrode portion is constructed from a second material having a second electrical conductivity different than the first electrical conductivity.

In some embodiments, a method includes inserting a catheter into a body such that an outer surface of an electrode is disposed against a target tissue. The electrode includes a first electrode portion and a second electrode portion. The first electrode portion and the second electrode portion collectively form the outer surface. The second electrode portion includes an edge portion of the outer surface. A voltage is applied to the first electrode portion and the second electrode portion via an electrical lead to produce an electric field from the outer surface. The first electrode portion and the second electrode portion are configured such that a ratio of a peak electric field strength at a central portion of the outer surface to a peak electric field strength at the edge portion of the outer surface is less than about 1.8.

As used in this specification and the appended claims, the singular forms “a,” “an” and “the” include plural referents unless the context clearly dictates otherwise. Thus, for example, the term “a member” is intended to mean a single member or a combination of members, “a material” is intended to mean one or more materials, or a combination thereof. Furthermore, the words “a” or “an” and the phrase “one or more” may be used interchangeably.

As used herein, the words “proximal” and “distal” refer to direction closer to and away from, respectively, an operator of the medical device. Thus, for example, the end of a catheter or delivery device contacting the patient's body would be the distal end of the medicament delivery device, while the end opposite the distal end (i.e., the end operated by the user) would be the proximal end of the catheter or delivery device.

As used herein, the terms “about” and/or “approximately” when used in conjunction with numerical values and/or ranges generally refer to those numerical values and/or ranges near to a recited numerical value and/or range. For example, in some instances, “about 40 [units]” can mean within ±25% of 40 (e.g., from 30 to 50). In some instances, the terms “about” and “approximately” can mean within ±10% of the recited value. In other instances, the terms “about” and “approximately” can mean within ±9%, ±8%, ±7%, ±6%, ±5%, ±4%, ±3%, ±2%, ±1%, less than ±1%, or any other value or range of values therein or therebelow. The terms “about” and “approximately” may be used interchangeably.

In a similar manner, term “substantially” when used in connection with, for example, a geometric relationship, a numerical value, and/or a range is intended to convey that the geometric relationship (or the structures described thereby), the number, and/or the range so defined is nominally the recited geometric relationship, number, and/or range. For example, two structures described herein as being “substantially parallel” is intended to convey that, although a parallel geometric relationship is desirable, some non-parallelism can occur in a “substantially parallel” arrangement. Such tolerances can result from manufacturing tolerances, measurement tolerances, and/or other practical considerations (such as, for example, minute imperfections, age of a structure so defined, a pressure or a force exerted within a system, and/or the like). As described above, a suitable tolerance can be, for example, of ±1%, ±2%, ±3%, ±4%, ±5%, ±6%, ±7%, ±8%, ±9%, or ±10% of the stated geometric construction, numerical value, and/or range. Furthermore, although a numerical value modified by the term “substantially” can allow for and/or otherwise encompass a tolerance of the stated numerical value, it is not intended to exclude the exact numerical value stated.

While numerical ranges are provided for certain quantities, it is to be understood that these ranges can include all subranges therein. Thus, the range “from 50 to 80” includes all possible ranges therein (e.g., 51-79, 52-78, 53-77, 54-76, 55-75, 70-80, etc.). Furthermore, all values within a given range may be an endpoint for the range encompassed thereby (e.g., the range 50-80 includes the ranges with endpoints such as 55-80, 50-75, etc.).

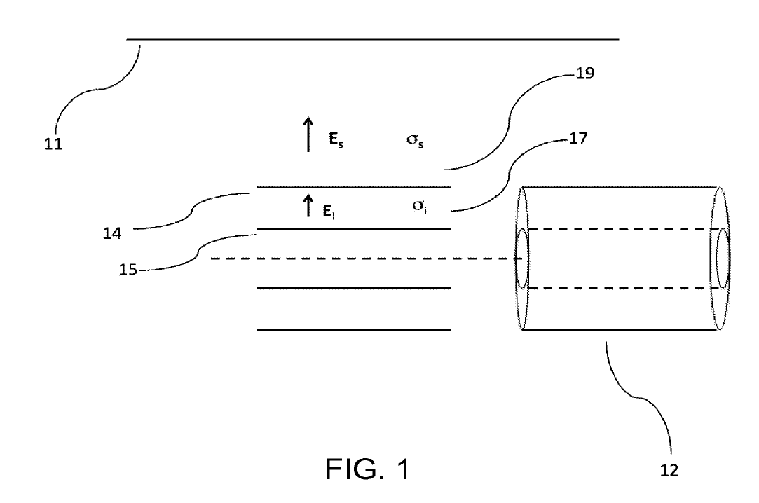

Referring to FIG. 1, consider current flowing out from an electrode 12 having a higher potential to a generally distant, lower potential surface 11. As shown in FIG. 1, the electrode 12 has a higher electric potential and the surface 11 is a lower potential surface (the surface 11 could be another electrode, an electrode patch or any other suitable surface with lower potential). The current flows from the electrode 12 through the intervening tissue space or region 19, and to the surface 11. The electrode 12 is ring-shaped for illustrative purposes, and is also schematically represented on the left side of FIG. 1 as having an inner surface 15 and an outer surface 14. In use, a voltage is applied to the inner electrode surface 15 so that it is at a higher electric potential relative to the ground or lower potential surface 11. This voltage at the inner electrode surface 15 produces and/or drives an electric current through the electrode 12 and subsequently through the tissue region 19. In the annular region 17 defined by the electrode material, the electrode material has electrical conductivity σᵢ and the electric field in that region is denoted by Eᵢ. In the tissue region 19 just outside the electrode outer surface 14, the electrical conductivity is σₛ and the electric field is Eₛ. As initial approximation, the current densities in the respective regions as constant, and thus the current density within the electrode 12 adjacent the outer surface 14 (jᵢ=σᵢEᵢ) is equal to the current density in the tissue region just outside the electrode (jₛ=σₛEₛ). Thus, the electric field magnitude just outside the electrode 12 (i.e., within the tissue region 19) is given by the equation:

$${E}_{s} = \frac{{σ}_{i}}{{σ}_{s}} {E}_{i} \qquad ( 1 )$$

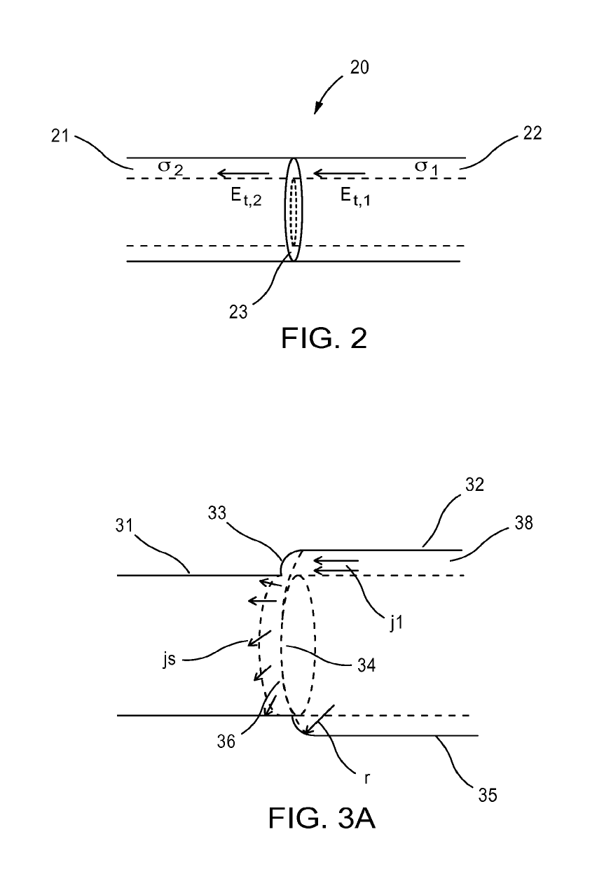

FIG. 2 shows a schematic view of a ring-shaped electrode 20, according to an embodiment. The electrode 20 includes and/or is constructed from two different materials: a first material in a left (or first) electrode portion 21 (with electrical conductivity σ₁) and a second material in a right (or second) electrode portion 22 (with electrical conductivity σ₂). The electrode cross section is an annular section, and the first electrode portion 21 is joined with the second electrode portion 22 as indicated by the perspective cross-sectional view of intersection 23. Similarly stated, the first electrode portion 21 and the second electrode portion 22 are distinct portions that form a non-homogenous electrode 20. The intersection 23 (or coupling) between the first electrode portion 21 and the second electrode portion 22 is smooth and/or continuous. Similarly stated, the first electrode portion 21 and the second electrode portion 22 are coupled together such that the outer diameter of the ring electrode 20 is constant, the outer surface of the ring electrode 20 is substantially continuous, and/or the annular area between the first electrode portion 21 and the second electrode portion 22 is substantially free from discontinuities. In this manner, when a voltage is applied to the electrode 20, the total (longitudinal) current through the annular cross section just to the left of section 23 and just to the right of section 23 is approximately equal. Since the area of cross section is the same on both sides of the intersection 23, the current densities on both sides (uniform in the cross section to a first approximation) is also equal. Thus, the current density in the first electrode portion 22 is equal to the current density in the second electrode portion 23 (j₁=σ₁E₁=j₂=σ₂E₂). Rearranging to solve for the electrical field results in:

$${E}_{2} = \frac{{σ}_{1}}{{σ}_{2}} {E}_{1} \qquad ( 2 )$$

where E₁ and E₂ are the electric field magnitudes in the first electrode portion 22 and second electrode portion 23, respectively. Thus, the multi-material (or composite) electrode can produce electrical fields having different magnitude based on the material properties (e.g., conductivity) of the different materials used.

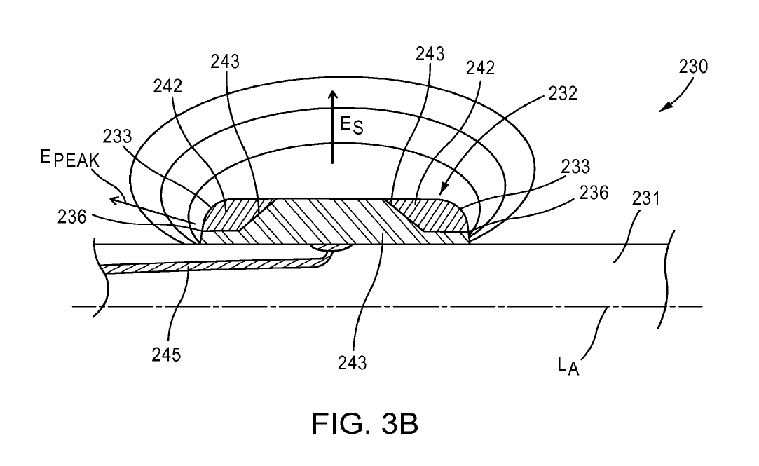

FIG. 3A is an illustration of an annular electrode 32 according to an embodiment coupled to and/or abutting a catheter shaft 31 constructed from an insulator. The electrode 32 has an annular region 38 bounded by an outer surface 35, an end surface 36, and an edge 33 between the outer surface 35 and the end surface 36. The end surface 36 is coupled to the catheter shaft 31 by any suitable means. The edge 33 has a rounded profile, as shown in FIG. 3A, with an associated radius of curvature having a value r. Thus, the end surface 36 and/or the edge 33 form an end boundary of the electrode 32. As shown, the edge 33 (with the curvature radius r) runs all the way around the circumference 34 of the electrode and has a total circumferential length L. As indicated in the figure, the current and/or current density (as indicated by the arrows j₁) within the annular region 38 of the electrode 32 just before (or proximal to) the edge 33 flows out of the curved edge 33 and the end surface 36, as indicated by arrows jₛ. The total current in this region is the current density multiplied by the area normal to the flow of the current. Equating the total current just before the edge 33 (i.e., within the annular region 38) with the total current flowing out of the edge, produces the following equation:

σₛEₛ(frL)=σ₁E₁A_{c} (3)

where f is a geometric factor (equal to π/4 for an edge that is quarter of a circle), σ₁ and E₁ represent electrical conductivity and electric field magnitude just within the electrode 32, σₛ and Eₛ represent electrical conductivity and electric field magnitude just outside the edge, and A_{c} is the (annular) area of cross section of the electrode. Equation (3) can be rewritten to obtain the following expression for the electric field Eₛ just outside of the boundary and/or edge 33 and end surface 36 of the electrode 32:

$${E}_{s} = \frac{{σ}_{1}}{{σ}_{s}} ( \frac{1}{frL} ) {E}_{1} {A}_{c} \qquad ( 4 )$$

The field E₁ is the longitudinal electric field just within the annular region 38 of the electrode 32. Equation (4) shows that the electric field Eₛ (just outside the electrode) is inversely proportional to the curvature radius of the edge, is inversely proportional to edge length (or circumference L), and is proportional to the electric field E₁ just inside the electrode, and to the ratio of inner and outer conductivities

$$\frac{{σ}_{1}}{{σ}_{s}}$$

From equation (4), it is apparent that for a given internal electric field E₁, the external electric field Eₛ can be reduced when the material that forms the edge 33 and/or the end surface 36 of the electrode is a conductor with a relatively smaller value of conductivity σ₁ (e.g. relative to other portions of the electrode), so that the ratio

$$\frac{{σ}_{1}}{{σ}_{s}}$$

is thereby reduced. However, for a given applied voltage and other factors remaining the same, simply using a lower conductivity material for the entire electrode correspondingly increases the internal electric field E₁, leading to the same external electric field Eₛ. Thus, in some embodiments as described herein, an electrode can include multiple different sections that can result in reduced external electric fields Eₛ near the electrode edges and/or boundaries.

For example, FIG. 3B shows a cross-sectional view of a portion of medical device 230 including an electrode 232 constructed from multiple different materials. Because the illustrated device 230 is symmetrical, the cross-sectional view shows only the portion of the device above a longitude axis A_{L}. In particular, the electrode 232 is coupled to a shaft 231, and is electrically coupled to a voltage source (not shown) via an electrical lead 245. The electrical lead 245 can be any suitable lead, such as an insulated lead including a high dielectric strength material (such as Teflon with an appropriate thickness) to be able to withstand high voltage pulses (e.g., up to 500 VDC) without dielectric breakdown. Although the lead 245 is shown as being coupled to the first electrode portion 241 (described below), in other embodiments, the lead can be coupled to any portion of the electrode 232.

The shaft 231 can be any suitable shaft, catheter and/or delivery device suitable for positioning the electrode 232 in proximity to and/or in contact with a target tissue. In this manner, as described herein, the medical device 230 can be used to deliver electrical impulse therapy to produce irreversible electroporation to treat any condition, such as cardiac arrhythmia.

As shown in FIG. 3B, the electrode 232 is a ring electrode having a first electrode portion 241 and a pair of second electrode portions 242 disposed at each end of the electrode 232. Similarly stated, the first electrode portion 241 is a central portion that is disposed between the two second electrode portions 242. The first electrode portion 241 is distinct from and/or non-homogeneous with the second electrode portion 242, and is coupled to the second electrode portion 242 at the interface 243. Although the interface 243 is shown as being tapered, in other embodiments, the interface between the first electrode portion 241 and the second electrode portion 242 can be substantially normal to a longitudinal axis L_{A} of the electrode 232 and/or the shaft 231. Similarly stated, although the outer diameter of the first electrode portion 241 at the interface 243 is shown as varying in a direction along the longitudinal axis L_{A}, in other embodiments, the outer diameter at the interface 243 can be constant and/or can vary in a discontinuous manner (i.e., a step change forming the interface 243).

The first electrode portion 241 and the second electrode portion 242 collectively form an outer surface 235 from which an electric field Eₛ is produced when a voltage is applied to the electrode 232 (e.g., via the lead 245). The electric field is shown in FIG. 3B as the curved lines extending from the outer surface 235. As shown in FIG. 3B, the outer surface 235 is continuous, smooth and/or defines a substantially constant outer diameter of the electrode 232. Thus, the outer surface 235 is continuous even though the first electrode portion 241 and the second electrode portion 242 are distinct and/or separate portions having separate material properties, as described herein. In other embodiments, however, the portion of the outer surface formed by the first electrode portion 241 can be recessed from the portion of the outer surface formed by the second electrode portion 242. In yet other embodiments, the portion of the outer surface formed by the second electrode portion 242 can be recessed from the portion of the outer surface formed by the first electrode portion 241.

The second electrode portions 242 form at least a portion of each end surface 236, each of which is coupled to the shaft 231. The second electrode portions 242 also include a radiused edge 235. Similarly stated, each second electrode portion 242 includes a transition region between the substantially cylindrical outer surface 235 and the end surface 236. Thus, the second electrode portions 242 define the end boundaries of the electrode 232. As described above, the magnitude of the electric field produced in the region of the boundaries is influenced by the geometry thereof (i.e., the radius of curvature, the angle between the end surface 236 and the outer surface 235, and the like). Thus, when a voltage is applied to the electrode 232, regions of peak electrical field strength (identified as E_{PEAK} in FIG. 3B) generally occur at the boundaries or edges.

The first electrode portion 241 is constructed from and/or includes a first material having a first electrical conductivity. The second electrode portion 242 is constructed from and/or includes a second material having a second electrical conductivity different than the first electrical conductivity. In particular, the second electrical conductivity is less than the first electrical conductivity. In this manner, and in accordance with Equation (4), the magnitude of the external electric field E_{PEAK} in the regions adjacent the end surface 236 and/or edge 235 can be reduced when compared to that which would result for an electrode having a constant conductivity. Moreover, because the first electrode portion 241 has a higher conductivity, the ratio of magnitude of the external electric field E_{PEAK} and the magnitude the external electric field Eₛ in the region adjacent the cylindrical outer surface 235 is less than about 2. In other embodiments, the geometry of the edge 235 and/or the ratio of the thermal conductivity between the first electrode portion 241 and the second electrode portion 242 can be such that the ratio of E_{PEAK} and Eₛ is less than about 1.8, 1.5, or 1.25. In this manner, the device 230 can produce tissue ablation at a target tissue location, while leaving surrounding tissue relatively intact and unchanged. In particular, the device 230 can generate a local electric field in a tissue region that is large enough to drive irreversible electroporation in that region, while maintaining the peak electric field values below a predetermined threshold.

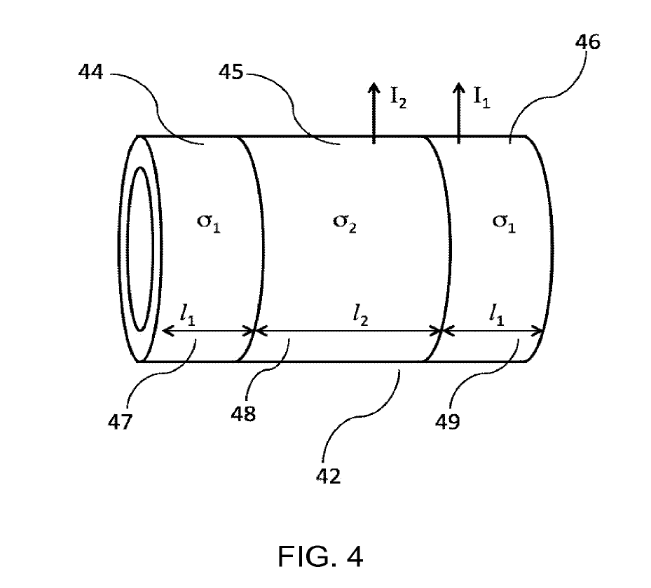

FIG. 4 shows a ring-shaped (with annular cross-section) electrode 42, including a higher electrical conductivity region 45 and two lower conductivity regions 44 and 46. The region 45 is constructed from and/or includes a material having electrical conductivity σ₂, and is flanked on either side by regions 44 and 46 constructed from and/or including a relatively lower electrical conductivity material with electrical conductivity σ₁ (so that σ₁<σ₂). Regions 44 and 46 have edges with an edge radius of curvature r (the curvature of the edges is not shown in FIG. 4) and an edge (circumferential) length L. As indicated in FIG. 4 by reference characters 47 and 49, regions 44 and 46 have identical lengths l₁, while region 45 has length l₂ as indicated by the reference character 48 (with l₂>l₁). The net current flowing out of outer surface of region 45 into surrounding tissue is denoted by l₂, while the net current flowing out of the outer surface of each of regions 44 and 46 is denoted by l₁. In some embodiments, the electrode 42 can be configured such that the major portion of current flows out of the central region 45 rather than the edge regions 44 and 46. As an approximation, if I is the total current flowing out of the electrode 42, the current flowing from the different portions of the outer surface can be represented by the equations:

$$\begin{matrix}{I}_{2} = I \frac{{σ}_{2} {l}_{2}}{( 2 {σ}_{1} {l}_{1} + {σ}_{2} {l}_{2} )} and & ( 5 ) \\ {I}_{1} = I \frac{{σ}_{1} {l}_{1}}{( 2 {σ}_{1} {l}_{1} + {σ}_{2} {l}_{2} )} & ( 6 )\end{matrix}$$

Writing I₁=j_{1,⊥}A_{c} with area of cross section A_{c} and transverse (i.e., perpendicular to the longitudinal axis of the electrode) current density j_{1,⊥}, equation (6) (representing the “edge” current) can also be written in terms of transverse electric field E_{1,⊥} as:

$${E}_{1 , ⊥} {A}_{c} = I \frac{{l}_{1}}{( 2 {σ}_{1} {l}_{1} + {σ}_{2} {l}_{2} )} \approx \frac{{Il}_{1}}{{σ}_{2} {l}_{2}} \qquad ( 7 )$$

where the electrode 42 is configured such that σ₂l₂>>2σ₁l₁.

The longitudinal electric field in regions 44 and 46 in FIG. 4 is approximately proportional to the transverse electric field (apart from factors involving geometry), and thus the longitudinal electric field is represented by:

$${E}_{1 , ‖} {A}_{c} ∝ \frac{{Il}_{1}}{{σ}_{2} {l}_{2}} \qquad ( 8 )$$

Using this result, the external electric field just outside the electrode edges of regions 44 and 46 can be written from equation (4) as

$${E}_{s} = \frac{{σ}_{1}}{{σ}_{s}} ( \frac{1}{frL} ) {E}_{1 , ‖} {A}_{c} ∝ \frac{{Il}_{1}}{{σ}_{s} {l}_{2}} \frac{{σ}_{1}}{{σ}_{s}} ( \frac{1}{frL} ) \qquad ( 9 )$$

where L is edge length or circumference and r is edge radius.

Comparing the above result for the composite or “multi-material” electrode 42 of the type shown in FIG. 4 with that for an electrode constructed from a single material (with electrical conductivity σ₂, and total length lₜₒₜ), in which case the external field E′ₛ near the electrode edges can be written (from an analysis similar to the above) as:

$${E}_{s}^{′} = \frac{{σ}_{2}}{{σ}_{s}} ( \frac{1}{frL} ) {E}_{1 , ‖} {A}_{c} ∝ \frac{{Il}_{1}}{{σ}_{s} {l}_{tot}} ( \frac{1}{frL} ) \qquad ( 10 )$$

Dividing equation (9) by equation (10) provides a ratio of the electric field strength near the edges of a composite (or multi-material) electrode (e.g., electrode 42) and a single material (or homogeneous) electrode:

$$\frac{{E}_{s}}{{E}_{s}^{′}} = \frac{{σ}_{1}}{{σ}_{2}} \frac{{l}_{tot}}{{l}_{2}} \qquad ( 11 )$$

Thus, the external edge electric field for the composite electrode can be reduced significantly compared to that of the single-material electrode by configuring the electrode (e.g., electrode 42 or any of the electrodes described herein) such that σ₂l₂>>σ₁lₜₒₜ. This would make

$$\frac{{E}_{s}}{{E}_{s}^{′}} ≪ 1$$

and would also satisfy the inequality mentioned with reference to and just after equation (7). In some embodiments, the electrode 42 (or any of the electrodes described herein) can be configured such that

$$\frac{{σ}_{2} {l}_{2}}{{σ}_{1} {l}_{tot}} > 3.$$

In other embodiments, the electrode 42 (or any of the electrodes described herein) can be configured such that

$$\frac{{σ}_{2} {l}_{2}}{{σ}_{1} {l}_{tot}} > 5.$$

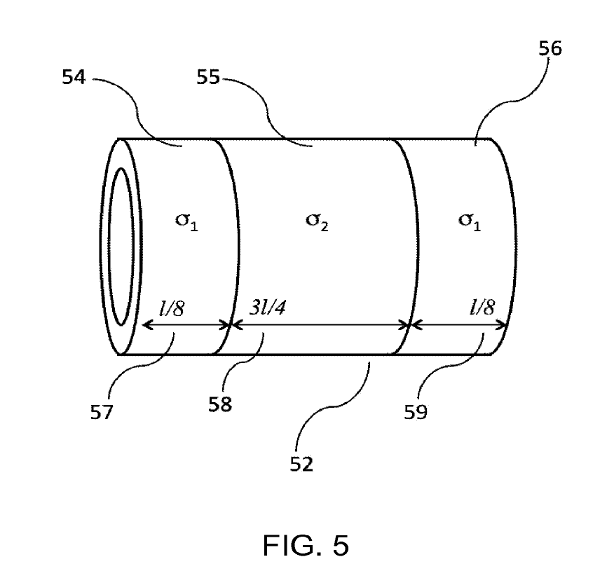

FIG. 5 shows an embodiment of a composite electrode 52 having a uniform and/or smooth lateral (or outer) surface and a total length l. The electrode 52 has a middle portion 55 having a length of 3 l/4 as indicated by the reference character 58. The middle portion 55 is constructed from a material with electrical conductivity σ₂. The electrode 52 includes end portions 54 and 56, each having a length l/8 as indicated by the reference characters 57 and 59, respectively. The end portions 54 and 56 are constructed from a material with electrical conductivity σ₁. The materials comprising the different electrode regions 55 and 54 can have a ratio of electrical conductivities so that the ratio

$$\frac{{σ}_{2} {l}_{2}}{{σ}_{1} {l}_{tot}}$$

is at least 3. In some embodiments, the ratio of electrical conductivities is at least about 3, at least about 4, or at least about 5. In some embodiments, the ratio of

$$\frac{{σ}_{2} {l}_{2}}{{σ}_{1} {l}_{tot}}$$

is at least about 4, at least about 5, or at least about 6.

The electrode materials from which the middle portion 55 and the end portions 54 and 56 (as well as any other electrode portions shown and described herein) can be any suitable biocompatible materials. For purely illustrative purposes, examples of biocompatible materials for the higher electrical conductivity and lower electrical conductivity electrode regions include, respectively, silver and palladium

$$( \frac{{σ}_{2}}{{σ}_{1}} ≅ 6.5 ) ,$$

silver and stainless steel

$$( \frac{{σ}_{2}}{{σ}_{1}} ≅ 47 ) ,$$

silver and platinum

$$( \frac{{σ}_{2}}{{σ}_{1}} ≅ 6.7 ) ,$$

platinum and titanium

$$( \frac{{σ}_{2}}{{σ}_{1}} ≅ 3.9 ) ,$$

platinum and stainless steel

$$( \frac{{σ}_{2}}{{σ}_{1}} ≅ 7 ) ,$$

and any other suitable combinations thereof. In other embodiments, the electrode 52 and any other electrodes described herein can include the platinum-iridium alloys or titanium instead of platinum, gold instead of silver, and any other suitable substitutions and/or combinations thereof.

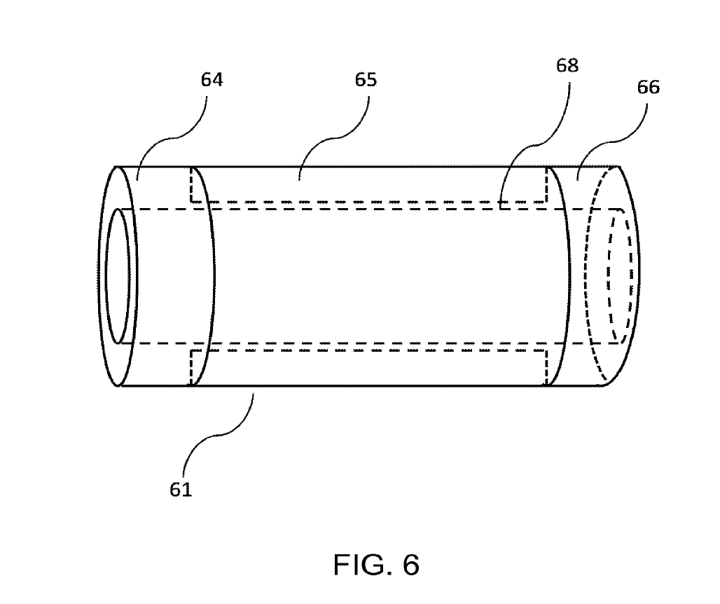

The different materials of any of the electrodes shown and described herein can be joined together in any suitable fashion. For example, FIG. 6 illustrates a composite electrode according to an embodiment in the form of a cylindrical annular electrode 61. The electrode 61 includes a midsection 65 constructed from a first material with a first electrical conductivity that is plated or deposited over a second material with a second electrical conductivity. As shown, the second material forms a thin layer or substrate 68 in the midsection 65 that expands in cross-sectional area to form the end sections 64 and 66. Other methods of construction can be employed. For example, in some embodiments, an electrode can be constructed by starting with a single thin ring of the second material with length equal to total electrode length, and then attaching to the outer surface thereof three rings of different materials. The three rings can include, respectively, the second material, the first material and the second material. The “outer rings” can be coupled to the substrate (e.g., substrate 68) using a variety of methods, such as fusing, annealing, plating, welding, crimping or lamination to ensure good electrical contact at all interfaces. Similarly stated, the interface between the different electrode portions of the electrode 61 and any of the electrodes described herein can be free of discontinuities, insulation layers and/or the like. The construction methods described here are for illustrative purposes only and one skilled in the art may devise various other methods of constructing the electrodes described herein.

In some embodiments, the thickness of the layer of first material in midsection 65 can be at least approximately equal to or greater than the thickness of the substrate 68 of second material in the midsection. In some embodiments, the length of the midsection 65 is at least twice as large as the length of either of the end sections 64 and 66. Moreover, in some embodiments, the electrical conductivity of the first material is at least four times larger than the electrical conductivity of the second material. The electrode materials are chosen to be biocompatible, and can include any suitable materials, as described herein. For purely illustrative purposes, examples of biocompatible material choices for the higher electrical conductivity and lower electrical conductivity electrode regions include, respectively, silver and palladium

$$( \frac{{σ}_{2}}{{σ}_{1}} ≅ 6.5 ) ,$$

silver and stainless steel

$$( \frac{{σ}_{2}}{{σ}_{1}} ≅ 47 ) ,$$

silver and platinum

$$( \frac{{σ}_{2}}{{σ}_{1}} ≅ 6.7 ) ,$$

platinum and titanium

$$( \frac{{σ}_{2}}{{σ}_{1}} ≅ 3.9 ) ,$$

platinum and stainless steel

$$( \frac{{σ}_{2}}{{σ}_{1}} ≅ 7 ) ,$$

and any suitable combination thereof. Other examples include the choice of platinum-iridium alloys or titanium instead of platinum, gold instead of silver, and any other suitable combinations and substitutions.

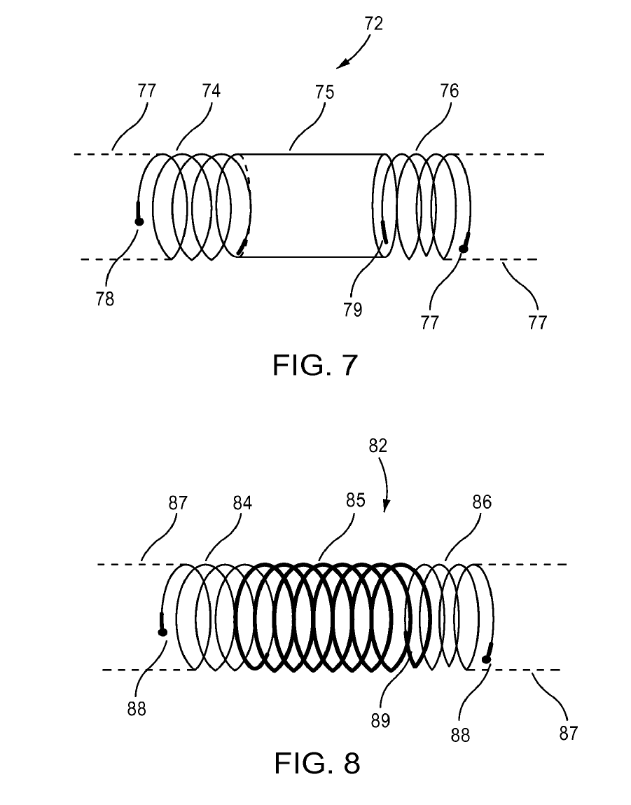

FIG. 7 illustrates a composite (or multi-section) electrode 72 according to an embodiment. The electrode 72 includes three segments 74, 75 and 76, with the midsection 75 including a rigid electrode portion in the form of a cylindrical annular electrode constructed from a first material with a first electrical conductivity (for clarity purposes, the annular structure is not shown in FIG. 7). The midsection 75 is flanked by, disposed between and/or surrounded by two end sections 74 and 76. The end sections 74 and 76 are in the form of windings, coils and/or springs that are capable of flexing and that are constructed from a second material with a second electrical conductivity. In some embodiments, the ends 78 and 79 of each flexible electrode portion 74 and 76 are rounded. As shown, the inside end 79 of each flexible electrode portion is attached to the rigid electrode portion 75 by local spot welding, laser welding or other suitable methods. In some embodiments, the outer ends 78 of the flexible electrode portions 74 and 76 can be covered and/or protected from exposure to the exterior of a catheter by being disposed within a polymeric thin-walled tube indicated schematically by the covering 77 in FIG. 7. In some embodiments, the axial length of the midsection 75 is at least twice as large as the axial length of either of the end sections 74 and 76, while the electrical conductivity of the first material is at least four times larger than the electrical conductivity of the second material. In some embodiments, the electrode materials are chosen to be biocompatible, using any of the materials described herein. For purely illustrative purposes, examples of biocompatible material choices for the higher electrical conductivity and lower electrical conductivity electrode segments or portions include, respectively, silver and palladium

$$( \frac{{σ}_{2}}{{σ}_{1}} ≅ 6.5 ) ,$$

silver and stainless steel

$$( \frac{{σ}_{2}}{{σ}_{1}} ≅ 47 ) ,$$

silver and platinum

$$( \frac{{σ}_{2}}{{σ}_{1}} ≅ 6.7 ) ,$$

platinum and titanium

$$( \frac{{σ}_{2}}{{σ}_{1}} ≅ 3.9 ) ,$$

platinum and stainless steel

$$( \frac{{σ}_{2}}{{σ}_{1}} ≅ 7 ) ,$$

and any suitable combinations thereof. Other examples include the choice of platinum-iridium alloys or titanium instead of platinum, gold instead of silver, and any suitable substitutions and/or combinations.

FIG. 8 illustrates a composite (or multi-section) electrode 82 according to an embodiment in the form of a completely flexible electrode. The electrode 82 includes three segments 84, 85 and 86, with the midsection 85 being a flexible electrode portion in the form of coils and/or springs that are capable of flexing. The midsection 85 is constructed from a first material with a first electrical conductivity (indicated by a thicker line in FIG. 8). The midsection 85 is flanked by, disposed between and/or surrounded by the two end sections 84 and 86. The two end section 84 and 86 are in the form of coils or springs that are capable of flexing, and are constructed from a second material with a second electrical conductivity (the second material is indicated by a thinner line in FIG. 8). In some embodiments, the ends 88 of each flexible electrode 84 and 86 are rounded. The inside end 89 of each flexible electrode is smoothly and/or continuously attached to a respective outer end of the midsection electrode 85 by local spot welding, laser welding or other suitable methods. In some embodiments, the outer ends 88 of the flexible electrodes can further be covered and/or protected from exposure to the exterior of a catheter by being disposed within a polymeric thin-walled tube indicated schematically by 87 in FIG. 8. In some embodiments, the axial length of the midsection electrode portion 85 is at least twice as large as the length of either of the end sections 84 and 86, while the electrical conductivity of the first material is at least four times larger than the electrical conductivity of the second material. In some embodiments, the electrode materials are chosen to be biocompatible, using any of the materials described herein. For purely illustrative purposes, examples of biocompatible material choices for the higher electrical conductivity and lower electrical conductivity electrode segments or portions include respectively silver and palladium

$$( \frac{{σ}_{2}}{{σ}_{1}} ≅ 6.5 ) ,$$

silver and stainless steel

$$( \frac{{σ}_{2}}{{σ}_{1}} ≅ 47 ) ,$$

silver and platinum

$$( \frac{{σ}_{2}}{{σ}_{1}} ≅ 6.7 ) ,$$

platinum and titanium

$$( \frac{{σ}_{2}}{{σ}_{1}} ≅ 3.9 ) ,$$

platinum and stainless steel

$$( \frac{{σ}_{2}}{{σ}_{1}} ≅ 7 ) ,$$

and any suitable combinations thereof. Other examples include the choice of platinum-iridium alloys or titanium instead of platinum, gold instead of silver, and any suitable substitutions and/or combinations.

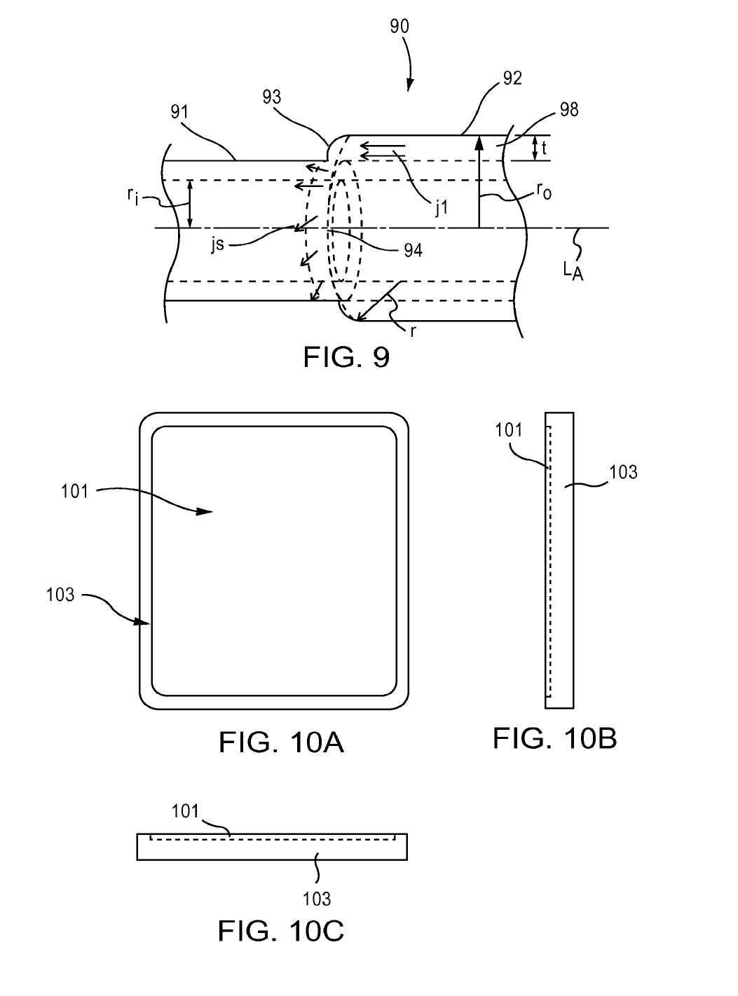

Although the electrode 230 is shown and described above as having an outer surface of constant diameter, in other embodiments, an electrode can be constructed of multiple materials joined together thereby producing a surface having a portion that is recessed. For example, FIG. 9 is an illustration of a portion of a composite (or multi-material) annular electrode 90 according to an embodiment. The electrode 90 includes a first annular electrical conductor 92 abutting and/or coupled to a second annular electrical conductor 91 that is distinct and/or separate from the first electrical conductor. Similarly stated, the first conductor (or electrode portion) 92 is different from and/or non-homogeneous with the second conductor (or electrode portion) 91. The first electrode portion 92 has an edge 93, assumed to have a rounded profile as shown in FIG. 9, with an associated radius of curvature having a value r. The edge 93 (with this curvature radius) runs all the way around the circumference 94 of the first electrode portion 92 and has a total length L (the circumferential length). Thus, the end surface and/or the edge 93 form an end boundary of the first electrode portion 92. As indicated in FIG. 9, the current density (as indicated by the arrows j₁) within the annular region 98 of the electrode 90 just before (or proximal to) the edge 93 flows out of the curved edge 93 as indicated by arrows jₛ. In some embodiments, the annular region 98 is thin relative to the electrode radius rₒ (the radius of the outer cylindrical surface). For example, the (radial) thickness of the annular region 98 is identified as having a thickness t, so that the annular cross-sectional area of the annular region 98 is approximately tL. As a fraction of the total (annular) cross section A_{c} it can be written as tA_{c}/rᵢ, where rᵢ is the inner electrode radius. To a first approximation, the total current in the annular region 98 of the electrode just before (or proximal to) the edge 93 can be equated with the total current flowing out of the edge 93:

$${σ}_{s} {E}_{s} ( frL ) \approx {σ}_{1} {E}_{1} \frac{t}{{r}_{i}} {A}_{c} \qquad ( 12 )$$

where f is a geometric factor (equal to π/4 for an edge that is quarter of a circle), σ₁ and E₁ represent electrical conductivity and electric field magnitude just within the electrode, and σₛ and Eₛ represent electrical conductivity and electric field magnitude just outside the edge. Equation (12) can be rewritten to obtain:

$${E}_{s} = \frac{{σ}_{1}}{{σ}_{2}} ( \frac{1}{frL} ) ( \frac{t}{{r}_{i}} ) {E}_{1} {A}_{c} \qquad ( 13 )$$

for the external electric field magnitude. The ratio t/r would typically be of order unity. If cross section area A_{c} is held approximately fixed and edge length L is varied, equation (13) shows that the external field Eₛ can be reduced by incorporating a large edge length L or edge transitions in the composite electrode.

Thus, in some embodiments, the electrode 90 and/or the first electrode portion 92 (or any of the other electrodes described herein) can include, for example, a wavy edge, multiple edges, etc. In some embodiments, the electrode portions 91 and 92, which are portions with relatively recessed or relatively raised profiles as shown in FIG. 9, can have different electrical conductivities. Thus, the electrode portion 91 is constructed from a first material with a first electrical conductivity while electrode portion 92 is constructed from a second material with a second electrical conductivity. To further reduce the external electric field Eₛ, in some embodiments, the electrode portion 92 with the raised profile (and with the second electrical conductivity) can have a smaller conductivity than the first electrical conductivity. Similarly stated, in some embodiments, the relatively more electrically conductive material is recessed. In some embodiments, the electrical conductivity of the first material is at least three times larger than the electrical conductivity of the second material. For purely illustrative purposes, examples of biocompatible material choices for the higher electrical conductivity and lower electrical conductivity electrode materials or portions include respectively silver and palladium

$$( \frac{{σ}_{2}}{{σ}_{1}} ≅ 6.5 ) ,$$

silver and stainless steel

$$( \frac{{σ}_{2}}{{σ}_{1}} ≅ 47 ) ,$$

silver and platinum

$$( \frac{{σ}_{2}}{{σ}_{1}} ≅ 6.7 ) ,$$

platinum and titanium

$$( \frac{{σ}_{2}}{{σ}_{1}} ≅ 3.9 ) ,$$

platinum and stainless steel

$$( \frac{{σ}_{2}}{{σ}_{1}} ≅ 7 ) ,$$

and any suitable combinations thereof. Other examples include the choice of platinum-iridium alloys or titanium instead of platinum, gold instead of silver, and any suitable substitutions and/or combinations thereof.

Although the electrode 90 is shown as being a ring electrode (i.e., forming a cylindrical outer surface), in other embodiments, a multi-material and/or composite electrode can be of any suitable shape. For example, FIGS. 10A-10C illustrate schematically, in three views, a composite electrode 100 according to an embodiment. The electrode 100 is in the form of a relatively thin, planar electrode constructed from two materials with differing properties of electrical conductivity. The portion 101 is surrounded by the portion 103. Moreover, the portion 103 forms an edge and/or boundary of the electrode 100. The electrical conductivity of portion 101 is greater than that of portion 103, and the two materials are joined together to be in electrical contact, as described herein. Portion 101 is inset and/or recessed from the edge of the electrode to provide a boundary of the material of portion 103 along the principal surface of the face. Portion 103 is predominantly exposed where the electrode local surface curvature is greatest (i.e., along the edge). The material of portion 101 is predominantly exposed where the surface curvature is least (i.e., the face). As shown, portions 101 and 103 share a common border. Relative to the edge portion 103 with lower electrical conductivity, the higher electrical conductivity region 101 is in the form of a recessed portion. For purely illustrative purposes, examples of biocompatible material choices for the higher electrical conductivity and lower electrical conductivity electrode portions include respectively silver and palladium

$$( \frac{{σ}_{2}}{{σ}_{1}} ≅ 6.5 ) ,$$

silver and stainless steel

$$( \frac{{σ}_{2}}{{σ}_{1}} ≅ 47 ) ,$$

silver and platinum

$$( \frac{{σ}_{2}}{{σ}_{1}} ≅ 6.7 ) ,$$

platinum and titanium

$$( \frac{{σ}_{2}}{{σ}_{1}} ≅ 3.9 ) ,$$

platinum and stainless steel

$$( \frac{{σ}_{2}}{{σ}_{1}} ≅ 7 ) ,$$

and any suitable combinations thereof. Other examples include the choice of platinum-iridium alloys or titanium instead of platinum, gold instead of silver, and any suitable substitutions or combinations thereof. This general type of composite electrode construction can result in a reduction of peak electric fields in spatial regions very close to the electrode. Although shown as being a substantially planar electrode, in other embodiments, the electrode 100 can be flexible, and can be wrapped about and/or coupled to a cylindrical member (e.g., a shaft) to form a substantially cylindrical electrode.

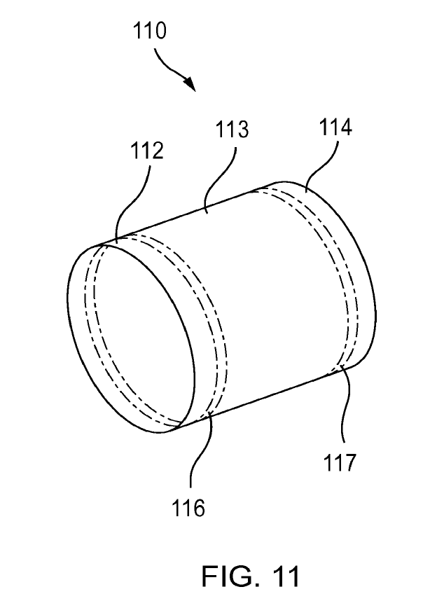

FIG. 11 illustrates a composite electrode 110 according to an embodiment, with segments of different materials. The electrode 110 includes a midportion 113 constructed from a first material with a first electrical conductivity. The midportion 113 flanked on either side by and/or disposed between end portions 112 and 114, respectively, that are constructed from a second material with a second electrical conductivity. In this embodiment the mid portion 113 has a profile (or outer surface) that is raised slightly (has larger diameter) relative to the end portions 112 and 114. In this manner, the electrode 110 can be similar to, for example, the electrode 90 described above.

A computational simulation was performed on the electrode 110 where the electric field distribution near the electrode was computed with a potential difference applied between the electrode and an exterior surface (not shown) in a conductive saline medium. The shading in FIG. 11 is a graphical depiction of the results, with the regions identified as 116 and 117 representing the electric field intensities at the transitions from the first material to the second material. As described herein, when a voltage is applied to the electrode 110, regions of peak electrical field strength generally occur at the boundaries. Thus, by comparing the simulation results for the multi-material electrode shown in FIG. 11, with that from a single-material electrode, the difference in the spatial variation of the electric field produced can be analyzed.

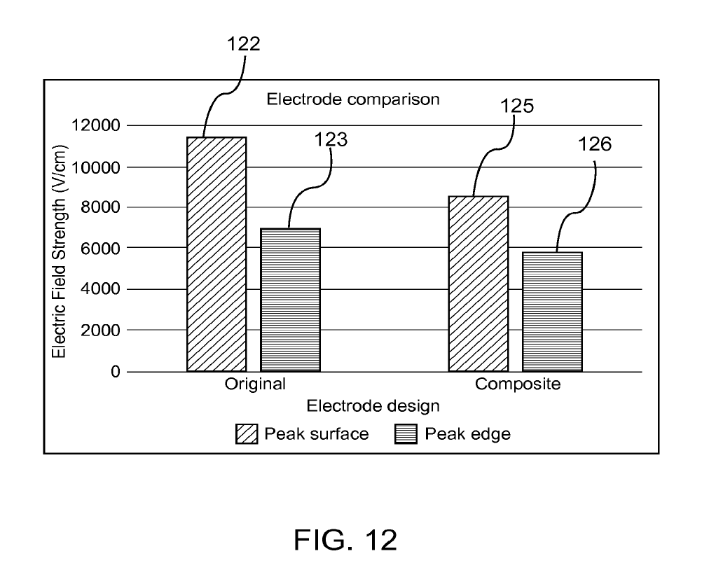

Specifically, FIG. 12 shows a graph of the simulation results comparing the peak “edge” electric field intensity and the peak “surface” electric field intensity for the electrode 110 and a single-material electrode having an annular cross section with the same inner diameter as the composite electrode of FIG. 11 and with the same outer diameter as that of the end portion 112 in FIG. 11 (i.e., having the same geometric construction). As shown, the peak “edge” electric field intensity (i.e., that occurred at the material transitions or edges) for the electrode 110 was in the range of 8500 Volts/cm (see the bar identified by reference character 125). The largest electric field intensity at the surface or lateral sides (e.g., the midsection 113) of the composite electrode 110 was approximately 5800 Volts/cm (see the bar identified by reference character 126). Thus, the ratio between the peak electric field at the edges to the peak electric field at the midsection 113 is on the order of 1.46. In comparison, the peak electric field intensity value at the edges of a single-material electrode comprising the first material and with similar overall dimensions was approximately 11,400 Volts/cm (see the bar identified by reference character 122). The largest electric field intensity at the surface or lateral sides of the single-material electrode was approximately 7000 Volts/cm (see the bar identified by reference character 123). Thus, the ratio between the peak electric field at the edges to the peak electric field at the midsection 113 is on the order of 1.63. The higher ratio indicates a greater spatial variability in the electric field strength, which can be undesirable in certain situations. As shown, the composite or multi-material electrode construction produced a reduction in peak electric field (when compared to the single-material electrode) of about 25% (from about 11,400 Volts/cm to 8500 Volts/cm). Likewise, the largest electric field intensity at the surface or lateral sides of the first material was approximately 8500 Volts/cm for the single-material electrode and approximately 5800 Volts/cm for the composite electrode construction.

It should be noted that one or more composite (or multi-material) electrodes in any of the embodiments disclosed herein and variations thereof can be incorporated on any suitable medical device, such as those devices described in International Patent Publication No. WO2014/025394 entitled “Catheters, Catheter Systems, and Methods for Puncturing Through a Tissue Structure,” which is incorporated herein by reference in its entirety. For example, FIG. 13 is an illustration of the distal portion 132 of a flexible medical device such as a catheter showing composite electrodes 133, 134 and 135 disposed at axial intervals along the distal portion. While three electrodes are shown, it should be noted that any number of composite electrodes in various embodiments as shown and described herein can be utilized on the medical device. Indeed a multiplicity of electrodes could include different combinations of the embodiments disclosed herein and their variations, so that, for example, some of the electrodes could be rigid composite electrodes, while some others could comprise flexible composite electrodes, and so on without limitations. Furthermore, a range of materials can be utilized in the composite electrode construction, as disclosed herein. Electrical leads (not shown) connect internally to the electrodes 133, 134 and 135. The leads are suitably insulated with high dielectric strength material (such as Teflon with an appropriate thickness) to be able to withstand high voltage pulses without dielectric breakdown.

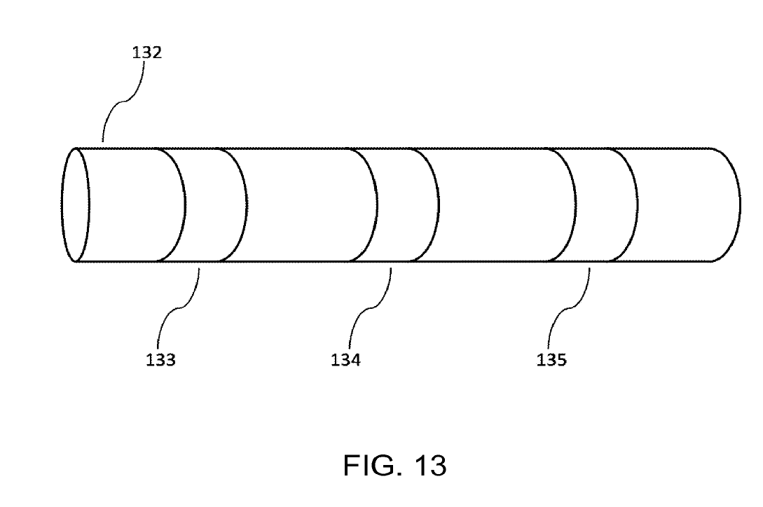

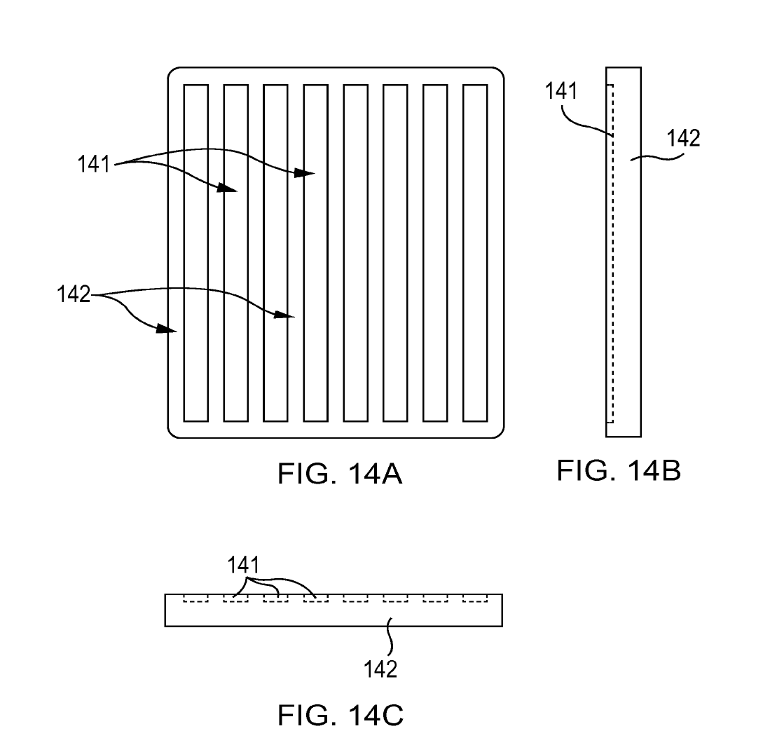

FIGS. 14A-C illustrates schematically, in three views, a composite electrode embodiment in the form of a relatively thin, planar electrode constructed from two materials with differing properties of electrical conductivity distributed as a multiplicity of distinct portions. The portion 142 surrounds a series of “islands” of portions 141, and a part of portion 142 forms an edge and/or boundary of the electrode. The two portions 141 and 142 respectively comprise distinct materials with different electrical conductivities. The electrical conductivity of (lighter shaded) portions 141 is greater than that of (darker shaded) portions 142, and the two materials are joined together to be in electrical contact. Portions 141 are inset and/or recessed from the edge of the electrode to provide a multiplicity of boundaries at the material of portions 142 along the principal surface of the face. Portions 142 are predominantly exposed where the electrode local surface curvature is greatest (the edge). The material of portions 141 is predominantly exposed where the surface curvature is least (the face). As shown in the figure, portions 141 and 142 share a multiplicity of common borders. Relative to the edge portions 142 with lower electrical conductivity, the higher electrical conductivity regions 141 are in the form of recessed portions. For purely illustrative purposes, examples of biocompatible material choices for the higher electrical conductivity and lower electrical conductivity electrode portions include respectively silver and palladium

$$( \frac{{σ}_{2}}{{σ}_{1}} ≅ 6.5 ) ,$$

silver and stainless steel

$$( \frac{{σ}_{2}}{{σ}_{1}} ≅ 47 ) ,$$

silver and platinum

$$( \frac{{σ}_{2}}{{σ}_{1}} ≅ 6.7 ) ,$$

platinum and titanium

$$( \frac{{σ}_{2}}{{σ}_{1}} ≅ 3.9 ) ,$$

platinum and stainless steel

$$( \frac{{σ}_{2}}{{σ}_{1}} ≅ 7 ) ,$$

and any suitable combinations thereof. Other examples include the choice of platinum-iridium alloys or titanium instead of platinum, gold instead of silver, and any suitable combinations or substitutions thereof. This general type of composite electrode construction comprising a large boundary length between at least two distinct materials with respectively lower electrical conductivity and higher electrical conductivity can result in a reduction of peak electric fields in spatial regions very close to the electrode.

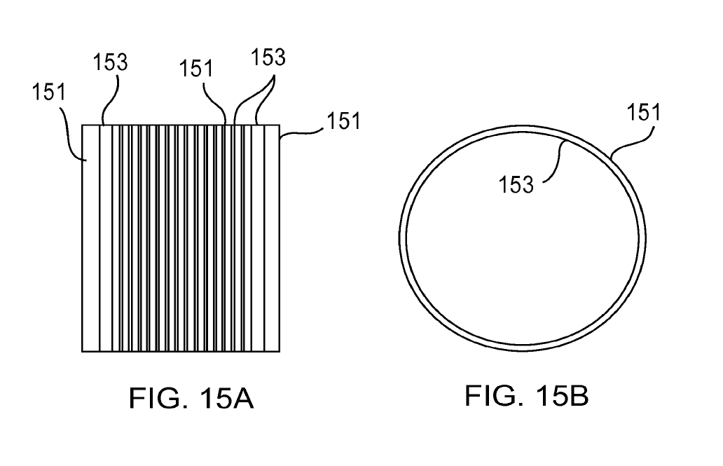

FIGS. 15A and 15B are a front view and a side view, respectively, of a composite electrode construction in the form of an electrode ring (or “ring electrode”) including a multiplicity of regions or portions with distinct electrical conductivities. The portions 151 form an edge and/or boundary of the electrode enclosing a set of “island” regions 153 within. Portions 151 (shaded light) of a first material with a first electrical conductivity are disposed at the edges of the electrode in the form of rings as shown, and alternate with ring-like portions 153 (shaded dark) of a second material with a second electrical conductivity. The portions 153 are slightly recessed (i.e., the rings have a slightly smaller diameter) relative to the portions 151. The second material comprising portions 153 is chosen to have a higher electrical conductivity than the first material comprising portions 151. It is apparent that in effect, this construction provides a large net or total boundary length between portions 151 and 153. As discussed in the foregoing, the electric field intensities close to the electrode can thereby be reduced, minimizing the likelihood of flash arcing. For purely illustrative purposes, examples of biocompatible material choices for the higher electrical conductivity and lower electrical conductivity electrode portions include respectively silver and palladium

$$( \frac{{σ}_{2}}{{σ}_{1}} ≅ 6.5 ) ,$$

or silver and stainless steel

$$( \frac{{σ}_{2}}{{σ}_{1}} ≅ 47 ) ,$$

silver and platinum

$$( \frac{{σ}_{2}}{{σ}_{1}} ≅ 6.7 ) ,$$

platinum and titanium

$$( \frac{{σ}_{2}}{{σ}_{1}} ≅ 3.9 ) ,$$

platinum and stainless steel

$$( \frac{{σ}_{2}}{{σ}_{1}} ≅ 7 ) ,$$

and any suitable combinations thereof. Other examples include the choice of platinum-iridium alloys or titanium instead of platinum, gold instead of silver, and any suitable combinations or substitutions. A variety of methods of construction can be employed as may be familiar to those skilled in the art. For example, one can start with a single thin ring of the second material with length equal to total electrode length, and attach over it rings comprising an alternating pattern of second material, first material, second material and so on using a variety of methods such as fusing, annealing, plating, welding, crimping or lamination to ensure good electrical contact at all interfaces. The construction methods described here are for illustrative purposes only. In other embodiments, and suitable methods of constructing the electrodes described herein can be employed.

It should be noted that a variety of alternate embodiments can be constructed, for example, in the form of a patterned surface wherein multiple regions of high electrical conductivity are disposed in slightly recessed fashion in the smaller-curvature portions of a composite electrode, and interspersed between multiple regions of low electrical conductivity disposed in relatively raised fashion in the larger-curvature portions. Such patterns can include without limitation stripes, dots, curvilinear shapes, fractal patterns and so on, as may be convenient for the construction and as may be optimal for a given application.

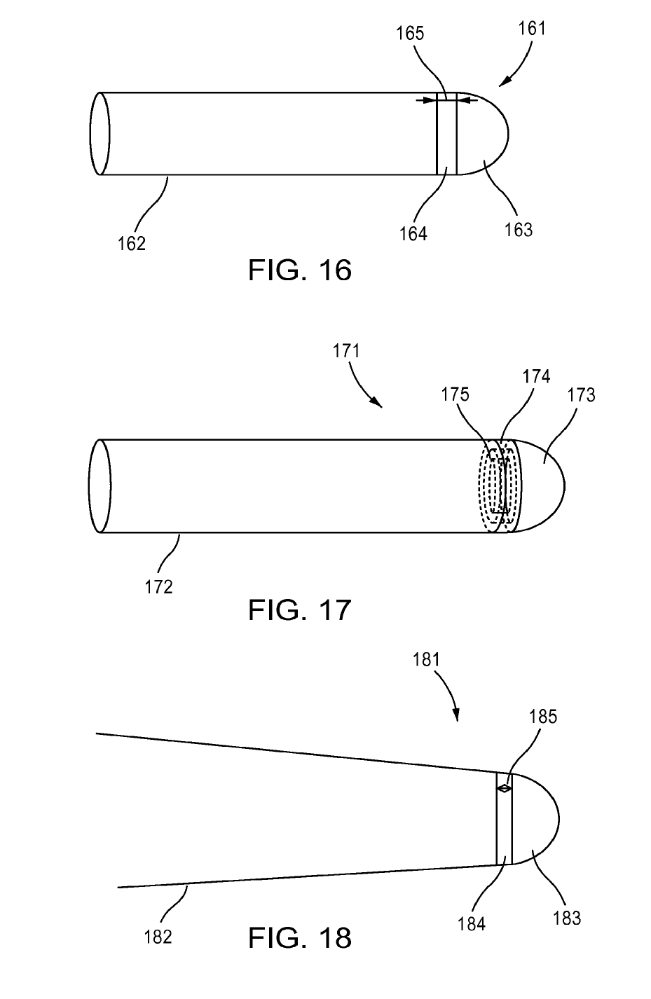

FIG. 16 is an illustration of a composite electrode 161 in the form of a tip electrode. As shown, the electrode 161 is located at the distal tip of a catheter or shaft. Specifically, the distal tip of a catheter shaft 162 includes a tip electrode 161 including a cap portion 163 and a ring portion 164. The portions 163 and 164 are smoothly and contiguously joined, as described herein. The ring portion 164 is constructed from a first material with a first electrical conductivity σ₁ while cap portion 163 is constructed from a second material with a second electrical conductivity σ₂. As shown, the cap portion 163 has a cross-sectional profile whose diameter varies along the longitudinal direction of the device. In this manner, the cap portion 163 forms a rounded and/or spherical end portion. The ring portion 164 is a substantially cylindrical shaped portion. In some embodiments, the radius of the ring portion 164 is at least twice as large as its width 165.

In some embodiments, the electrical conductivity of the second material is at least four times larger than the electrical conductivity of the first material. In other embodiments, the electrode materials are chosen to be biocompatible, and can include any suitable materials, as described herein. For examples biocompatible material choices for the higher electrical conductivity and lower electrical conductivity electrode portions include, respectively, silver and palladium

$$( \frac{{σ}_{2}}{{σ}_{1}} ≅ 6.5 ) ,$$

silver and stainless steel

$$( \frac{{σ}_{2}}{{σ}_{1}} ≅ 47 ) ,$$

silver and platinum

$$( \frac{{σ}_{2}}{{σ}_{1}} ≅ 6.7 ) ,$$

platinum and titanium

$$( \frac{{σ}_{2}}{{σ}_{1}} ≅ 3.9 ) ,$$

platinum and stainless steel

$$( \frac{{σ}_{2}}{{σ}_{1}} ≅ 7 ) ,$$

and any suitable combinations thereof. Other examples include the choice of platinum-iridium alloys or titanium instead of platinum, gold instead of silver, and other suitable substitutions and/or combinations thereof.

The smooth joining of the first material and second material can be accomplished by using a variety of methods such as fusing, annealing, plating, welding, crimping or lamination to ensure good electrical contact at all interfaces. The construction methods described here are for example purposes only and one skilled in the art may devise various other suitable methods of fabricating the electrodes described herein. The composite tip electrode described here can be a part of a focal ablation catheter that can be used in the treatment of a variety of clinical applications such as for example the delivery of ablation therapy for the treatment of Ventricular Tachycardia (VT). In such embodiments, the tip electrode (e.g., the electrode 161) is used in monopolar fashion and the ground electrode for the current return path could be a surface patch electrode placed on the patient exterior, or even an electrode or multiple electrodes on one or more different medical devices.

FIG. 17 illustrates a composite electrode 171 according to an embodiment in the form of a tip electrode. As shown, the electrode 171 is mounted at the distal tip of a catheter 172, and includes a cap portion 173 and a ring portion 174. The portions 173 and 174 are smoothly and contiguously joined. Portion 174 comprises a first material with a first electrical conductivity that is plated or deposited over a second material with a second electrical conductivity, the cap portion 173 also comprising the second material and with the second material forming a thin cylindrical layer or ring-like substrate portion 175 extending proximally from the cap portion 173. The ring portion 174 is then plated or otherwise deposited (for example, by a sputter deposition process) over the substrate portion 175. Other methods of construction can also be employed as may be familiar to those skilled in the art. As in the figure, the cap portion has a cross section profile whose diameter varies along the longitudinal direction of the device. In a preferred embodiment, the thickness of the layer of first material 174 can be at least approximately equal to or greater than the thickness of the substrate 175 of second material. In a preferred embodiment, the outer radius of the ring portion 174 is at least twice as large as its width, while the electrical conductivity of the second material is at least four times larger than the electrical conductivity of the first material. In a preferred embodiment, the electrode materials are chosen to be biocompatible, and a variety of material choices can be made by one skilled in the art. For purely illustrative purposes of providing examples, biocompatible material choices for the higher electrical conductivity and lower electrical conductivity electrode portions include respectively silver and palladium

$$( \frac{{σ}_{2}}{{σ}_{1}} ≅ 6.5 ) ,$$

or silver and stainless steel

$$( \frac{{σ}_{2}}{{σ}_{1}} ≅ 47 ) ,$$

silver and platinum

$$( \frac{{σ}_{2}}{{σ}_{1}} ≅ 6.7 ) ,$$

platinum and titanium

$$( \frac{{σ}_{2}}{{σ}_{1}} ≅ 3.9 ) ,$$

platinum and stainless steel

$$( \frac{{σ}_{2}}{{σ}_{1}} ≅ 7 ) ,$$

and any suitable combinations thereof. Other examples include the choice of platinum-iridium alloys or titanium instead of platinum, gold instead of silver, and other suitable substitutions and/or combinations.

FIG. 18 is an illustration of a composite electrode 181 according to an embodiment, in the form of a tip electrode. In this embodiment, the electrode 181 is located at the distal tip of a surgical instrument, possibly a handheld device, for the treatment of cardiac arrhythmias by tissue ablation with high voltage DC pulses or electrical energy. As shown, the distal portion 182 of a surgical instrument includes the rounded tip electrode 181 that includes a cap portion 183 and a ring portion 184. The distal portion 182 of the surgical instrument can have a taper, as shown. The portions 183 and 184 are smoothly and contiguously joined, as described herein. The ring portion 184 comprises a first material with a first electrical conductivity σ₁ while portion 183 comprises a second material with a second electrical conductivity σ₂. As in the figure, the cap portion has a cross section profile whose diameter varies along the longitudinal direction of the device. In a preferred embodiment, the radius of the ring portion 184 is at least twice as large as its width 185, while the electrical conductivity of the second material is at least four times larger than the electrical conductivity of the first material. In a preferred embodiment, the electrode materials are chosen to be biocompatible, and a variety of material choices can be made by one skilled in the art. For purely illustrative purposes of providing examples, biocompatible material choices for the higher electrical conductivity and lower electrical conductivity electrode portions include respectively silver and palladium

$$( \frac{{σ}_{2}}{{σ}_{1}} ≅ 6.5 ) ,$$

or silver and stainless steel

$$( \frac{{σ}_{2}}{{σ}_{1}} ≅ 47 ) ,$$

silver and platinum

$$( \frac{{σ}_{2}}{{σ}_{1}} ≅ 6.7 ) ,$$

platinum and titanium

$$( \frac{{σ}_{2}}{{σ}_{1}} ≅ 3.9 ) ,$$

platinum and stainless steel

$$( \frac{{σ}_{2}}{{σ}_{1}} ≅ 7 ) ,$$

and other suitable combinations thereof. Other examples include the choice of platinum-iridium alloys or titanium instead of platinum, gold instead of silver, and other suitable substitutions and/or combinations. The smooth joining of the first material and second material can be accomplished by using a variety of methods such as fusing, annealing, plating, welding, crimping or lamination to ensure good electrical contact at all interfaces. The construction methods described here are for example purposes only. In other embodiments, and suitable methods of constructing the electrode can be employed. The composite tip electrode described here can comprise part of a surgical instrument for focal ablation delivery that can be used in the treatment of a variety of clinical applications such as for example the delivery of ablation therapy for the treatment of Ventricular Tachycardia (VT); in this case the tip electrode is used in monopolar fashion and the ground electrode for the current return path could be a surface patch electrode placed on the patient exterior, or even an electrode or multiple electrodes on one or more different medical devices.

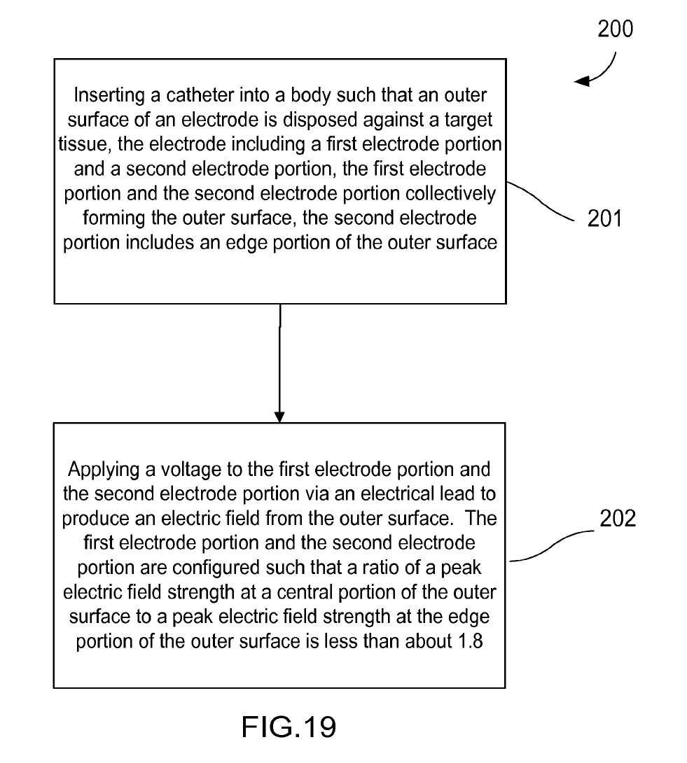

FIG. 19 is flowchart illustrating a method 200 of using a medical device according to an embodiment. As shown, the method 200 includes inserting a catheter into a body such that an outer surface of an electrode is disposed against a target tissue, at 201. The electrode includes a first electrode portion and a second electrode portion. The first electrode portion and the second electrode portion collectively form the outer surface. The second electrode portion includes an edge portion of the outer surface. The electrode can be any of the electrodes shown and described herein.

A voltage is applied to the first electrode portion and the second electrode portion via an electrical lead to produce an electric field from the outer surface, at 202. The first electrode portion and the second electrode portion are configured such that a ratio of a peak electric field strength at a central portion of the outer surface to a peak electric field strength at the edge portion of the outer surface is less than about 1.8. In other embodiments, the ratio of the peak electric field strength at the central portion of the outer surface to the peak electric field strength at the edge portion of the outer surface is less than about 1.7. In other embodiments, the ratio of the peak electric field strength at the central portion of the outer surface to the peak electric field strength at the edge portion of the outer surface is less than about 1.5.

In some embodiments, any of the electrodes described herein can be used in to deliver electrical impulse therapy to produce irreversible electroporation in conjunction with any suitable procedure, such as those described in International Patent Publication No. WO2014/025394 entitled “Catheters, Catheter Systems, and Methods for Puncturing Through a Tissue Structure,” which is incorporated herein by reference in its entirety. In such methods and systems, a DC voltage for electroporation can be applied to one or more electrodes coupled to a catheter. In some embodiments, all of the electrode sets of the catheter are activated simultaneously, while in other embodiments the electrode sets can be activated sequentially for voltage pulse application. The DC voltage can be applied to the electrodes in brief pulses sufficient to cause irreversible electroporation. The DC voltage applied to the electrode can be in the range of 0.5 kV to 10 kV, and more preferably in the range 1 kV to 4 kV, so that an appropriate threshold electric field is effectively achieved in the tissue to be ablated. The DC voltage pulse results in a current flowing between anode and cathode electrodes of the corresponding activated electrode set(s), with the current flowing through intervening tissue from the anode and returning back through the cathode electrode.

The time duration of each irreversible electroporation rectangular voltage pulse can be within the range from about 1 nanosecond to about 10 milliseconds. In other embodiments, the range can be between from 10 microseconds to about 1 millisecond, and/or within the range from about 50 microseconds to about 300 microseconds. The time interval between successive pulses of a pulse train could be in the range of about 10 microseconds to about 1 millisecond, within the range from about 50 microseconds to about 300 microseconds, or any other suitable range. The number of pulses applied in a single pulse train (with delays between individual pulses lying in the ranges just mentioned) can range from about 1 to about 100, and in some embodiments, within the range from 1 to 10. In some embodiments, a pulse train can be driven by a user-controlled switch or button, in one embodiment preferably mounted on a hand-held joystick-like device, while in an alternate embodiment it could be in the form of a computer mouse or other interface, or a foot pedal. Indeed, a variety of such triggering schemes can be implemented by those skilled in the art, as convenient for the application and without departing from the scope of the embodiments described herein. In one mode of operation a pulse train can be generated for every push of such a control button, while in an alternate mode of operation pulse trains can be generated repeatedly for as long as the user-controlled switch or button is engaged by the user.

The embodiments and devices described herein can be formed or constructed of one or more biocompatible materials. Examples of suitable biocompatible materials include metals, glasses, ceramics, or polymers. Examples of suitable metals include stainless steel, gold, titanium, platinum, silver, palladium, copper, nickel and/or alloys thereof. A polymer material may be biodegradable or non-biodegradable. Examples of suitable biodegradable polymers include polylactides, polyglycolides, polylactide-co-glycolides (PLGA), polyanhydrides, polyorthoesters, polyetheresters, polycaprolactones, polyesteramides, poly(butyric acid), poly(valeric acid), polyurethanes, and/or blends and copolymers thereof. Examples of non-biodegradable polymers include nylons, polyesters, polycarbonates, polyacrylates, polymers of ethylene-vinyl acetates and other acyl substituted cellulose acetates, non-degradable polyurethanes, polystyrenes, polyvinyl chloride, polyvinyl fluoride, poly(vinyl imidazole), chlorosulphonate polyolefins, polyethylene oxide, and/or blends and copolymers thereof.

Any of the first electrode portions or the second electrode portions described herein can be constructed from any suitable material having any suitable range of electrical conductivity. For example, any of the electrode portions described herein can be constructed from silver, palladium, stainless steel, titanium, platinum, nickel, and any alloys thereof.

The electrodes described herein can be constructed using any suitable procedures. In some embodiments, the electrode materials with chosen electrical conductivities can be plated, coated and/or otherwise applied in an appropriately thick layer on top of a different substrate material. In other embodiments, electrode portions can be coupled together using annealing, soldering, welding, crimping and/or lamination to ensure good electrical contact at all interfaces.

Any of the embodiments described herein can be used with any suitable devices, catheters and/or systems. Such can include any of the described in International Patent Publication No. WO2014/025394 entitled “Catheters, Catheter Systems, and Methods for Puncturing Through a Tissue Structure,” which is incorporated herein by reference in its entirety. Accordingly, the present electrode designs may be adapted for various procedures and/or uses, depending on the apparatus in which such electrodes are to be employed.

Although various embodiments have been described as having particular features and/or combinations of components, other embodiments are possible having a combination of any features and/or components from any of embodiments as discussed above. Where methods and/or schematics described above indicate certain events and/or flow patterns occurring in certain order, the ordering of certain events and/or flow patterns may be modified. Additionally certain events may be performed concurrently in parallel processes when possible, as well as performed sequentially. While the embodiments have been particularly shown and described, it will be understood that various changes in form and details may be made.

For example, although the electrodes described above are shown and described as being used to produce irreversible electroporation, in other embodiments, the electrodes and devices described herein can be used in conjunction with any suitable procedure.

Although the electrodes have been described herein as having specific shapes (e.g., a ring electrode, as shown in FIGS. 3A and 3B or a substantially planar electrode, as shown in FIG. 10A-10C), in other embodiments, any of the electrodes shown and described herein can have any suitable shape and/or size.

Although various embodiments have been described as having particular features and/or combinations of components, other embodiments are possible having a combination of any features and/or components from any of embodiments as discussed above.

For example, the electrical lead and connection shown and described in connection with the electrode 230 (FIG. 3B) can be used in any of the electrodes shown and described herein. As another example, the geometric proportions shown and described in connection with the electrode 52 (FIG. 5) can be used in any of the electrodes shown and described herein. As yet another example, the tapered joint between electrode portions shown and described in connection with the electrode 230 (FIG. 3B) can be used in any of the electrodes shown and described herein.

1-35. (canceled)

36. A method, comprising:
inserting a catheter into a body such that an outer surface of an electrode is disposed against a target tissue, the electrode including a first electrode portion and a second electrode portion, the first electrode portion and the second electrode portion collectively forming the outer surface, the second electrode portion includes an edge portion of the outer surface; and
applying a voltage to the first electrode portion and the second electrode portion via an electrical lead to produce an electric field from the outer surface, the first electrode portion and the second electrode portion configured such that a ratio of a peak electric field strength at a central portion of the outer surface to a peak electric field strength at the edge portion of the outer surface is less than about 1.8.

37. The method of claim 36, wherein the first electrode portion is constructed from a first material having a first electrical conductivity, and the second electrode portion is constructed from a second material, the second material having a second electrical conductivity less than the first electrical conductivity.

38. The method of claim 37, wherein a ratio of the first electrical conductivity to the second electrical conductivity is at least three.

39. The method of claim 36, wherein:
the electrode is a ring electrode; and
the outer surface is a cylindrical surface having a constant outer diameter.

40. The method of claim 36, wherein the second electrode portion includes an edge of the outer surface.

41. The method of claim 36, wherein the second electrode portion surrounds the first electrode portion and forms a boundary of the outer surface.

42. The method of claim 36, wherein:
the electrode is a ring electrode configured to be coupled to a catheter shaft;
the outer surface is a cylindrical surface of the ring electrode; and
the second electrode portion forms at least a portion of an end surface of the ring electrode, the end surface configured to be coupled to the catheter shaft.

43. The method of claim 36, wherein:
the electrode is a ring electrode;
the outer surface is a cylindrical surface of the ring electrode having a total length along a center line about which the cylindrical surface is defined; and
the first electrode portion forms a portion of the outer surface having a length at least 0.75 of the total length.

44. The method of claim 36, wherein:
the electrode is a ring electrode;
the outer surface is a cylindrical surface of the ring electrode;
the second electrode portion forms at least a portion of a first end surface of the ring electrode; and
the electrode includes a third electrode portion forming a portion of the cylindrical surface and at least a portion of a second end surface of the ring electrode.

45. The method of claim 36, wherein the first material is any one of platinum or silver and the second material is stainless steel.

46. The method of claim 36, wherein:
a first portion of the outer surface is formed by the first portion of the electrode; and
a second portion of the outer surface is formed by the second portion of the electrode, the first portion of the outer surface being recessed from the second portion of the outer surface.

47. The method of claim 36, wherein at least one of the first electrode portion or the second electrode portion includes a flexible coil.

48. An apparatus, comprising an electrode configured to be disposed against a target tissue during use, the electrode including:
a first electrode portion; and
a second electrode portion, the first electrode portion and the second electrode portion collectively forming an outer surface, the second electrode portion includes an edge portion of the outer surface,
the first electrode portion and the second electrode portion configured such that upon application of a voltage to the first electrode portion and the second electrode portion, a ratio of a peak electric field strength at a central portion of the outer surface to a peak electric field strength at the edge portion of the outer surface is less than about 1.8.

49. The apparatus of claim 48, wherein the first electrode portion is constructed from a first material having a first electrical conductivity, and the second electrode portion is constructed from a second material, the second material having a second electrical conductivity less than the first electrical conductivity.

50. The apparatus of claim 49, wherein a ratio of the first electrical conductivity to the second electrical conductivity is at least three.

51. The apparatus of claim 48, wherein:
the electrode is a ring electrode; and
the outer surface is a cylindrical surface having a constant outer diameter.

52. The apparatus of claim 48, wherein the second electrode portion includes an edge of the outer surface.

53. The apparatus of claim 48, wherein the second electrode portion surrounds the first electrode portion and forms a boundary of the outer surface.

54. The apparatus of claim 48, wherein the first material is any one of platinum or silver and the second material is stainless steel.

55. The apparatus of claim 48, wherein:
a first portion of the outer surface is formed by the first portion of the electrode; and
a second portion of the outer surface is formed by the second portion of the electrode, the first portion of the outer surface being recessed from the second portion of the outer surface.

56. The apparatus of claim 48, wherein at least one of the first electrode portion or the second electrode portion includes a flexible coil.
